(19) United States
(12) Patent Application Publication
(10) Pub. No.: US 2022/0301156 A1
(43) Pub. Date: Sep. 22, 2022

(54) METHOD AND SYSTEM FOR ANNOTATION EFFICIENT LEARNING FOR MEDICAL IMAGE ANALYSIS

(71) Applicant: Shenzhen Keya Medical Technology Corporation,, Shenzhen (CN)

(72) Inventors: Zhenghan Fang, Shoreline, WA (US); Junjie Bai, Seattle, WA (US); Youbing Yin, Kenmore, WA (US); Xinyu Guo, Redmond, WA (US); Qi Song, Seattle, WA (US)

(73) Assignee: Shenzhen Keya Medical Technology Corporation, Shenzhen (CN)

(21) Appl. No.: 17/591,758

(22) Filed: Feb. 3, 2022

Related U.S. Application Data
(60) Provisional application No. 63/161,781, filed on Mar. 16, 2021.

(51) Int. Cl. G06T 7/00; G16H 30/40

(52) U.S. Cl. CPC G06T 7/0012; G16H 30/40; G06T 2200/24; G06T 2207/20081; G06T 2210/12

(57) ABSTRACT

Embodiments of the disclosure provide systems and methods for analyzing medical images using a learning model. The system receives a medical image acquired by an image acquisition device. The system may additionally include at least one processor configured to apply the learning model to perform an image analysis task on the medical image. The learning model is trained jointly with an error estimator using training images comprising a first set of labeled images and a second set of unlabeled images. The error estimator is configured to estimate an error of the learning model associated with performing the image analysis task.

### CROSS-REFERENCE TO RELATED APPLICATION

This application claims the benefit of priority to U.S. Provisional Application No. 63/161,781, filed on Mar. 16, 2021, the entire content of which is incorporated herein by reference.

### TECHNICAL FIELD

The present disclosure relates to systems and methods for analyzing medical images, and more particularly systems and method for training an image analysis learning model with an error estimator for improving the performance of the learning model due to lack of labels in training images.

### BACKGROUND

Machine learning techniques have shown promising performance for medical image analysis. For example, machine learning models are used for segmenting or classifying medical images, or detecting objects, such as tumors, from the medical images. However, in order to obtain accurate machine learning models, i.e., models with low prediction errors, the training process usually requires large amounts of annotated data (e.g., labeled images) for training.

Obtaining the annotation for training is time-consuming and labor-intensive, especially for medical images. For example, in three-dimensional (3D) medical image segmentation problems, voxel-level annotation needs to be obtained, which is extremely time consuming, especially for high-dimensional and high-resolution volumetric medical images such as thin-slice CT. In addition, boundaries of the segmentation targets are often irregular and ambiguous, which makes detailed voxel-level delineation challenging even for experienced radiologists. For example, diseased regions such as pneumonia lesions in lung have irregular and ambiguous boundaries. Therefore, there is an unmet need for a learning framework for medical image analysis with low annotation cost.

Embodiments of the disclosure address the above problems by providing methods and systems for training an image analysis learning model with an error estimator for augmenting the labeled training images, thus improving the performance of the learning model

### SUMMARY

Novel systems and methods for training learning models for analyzing medical images with an error estimator and applying the trained models for image analysis are disclosed.

In one aspect, embodiments of the disclosure provide a system for analyzing medical images using a learning model. The system may include a communication interface configured to receive a medical image acquired by an image acquisition device. The system may additionally include at least one processor configured to apply the learning model to perform an image analysis task on the medical image. The learning model is trained jointly with an error estimator using training images comprising a first set of labeled images and a second set of unlabeled images. The error estimator is configured to estimate an error of the learning model associated with performing the image analysis task.

In another aspect, embodiments of the disclosure also provide a computer-implemented method for analyzing medical images using a learning model. The method may include receiving, by a communication interface, a medical image acquired by an image acquisition device. The method may also include applying, by at least one processor, the learning model to perform an image analysis task on the medical image. The learning model is trained jointly with an error estimator using training images comprising a first set of labeled images and a second set of unlabeled images. The error estimator is configured to estimate an error of the learning model associated with performing the image analysis task.

In yet another aspect, embodiments of the disclosure further provide a non-transitory computer-readable medium having a computer program stored thereon. The computer program, when executed by at least one processor, performs a method for analyzing medical images using a learning model. The method may include receiving a medical image acquired by an image acquisition device. The method may also include applying the learning model to perform an image analysis task on the medical image. The learning model is trained jointly with an error estimator using training images comprising a first set of labeled images and a second set of unlabeled images. The error estimator is configured to estimate an error of the learning model associated with performing the image analysis task

In some embodiments, the learning model and the error estimator may be trained by: training an initial version of the learning model and an error estimator with the first set of labeled images; applying the error estimator to the second set of unlabeled images to determine respective errors associated with the unlabeled images; determining a third set of labeled images from the second set of unlabeled images based on the respective errors; and training an updated version of the learning model with the first set of labeled images combined with the third set of labeled images.

In some embodiments, the image analysis task is an image segmentation task, and the learning model is configured to predict a segmentation mask. The error estimator is accordingly configured to estimate an error map of the segmentation mask.

In some embodiments, the image analysis task is an image classification task, the learning model is configured to predict a classification label. The error estimator is accordingly configured to estimate a classification error between the classification label predicted by the learning model and a ground-truth label included in a labeled image.

In some embodiments, the image analysis task is an object detection task, the learning model is configured to detect an object from the medical image, e.g., by predicting a bounding box surrounding the object and a classification label of the object. The error estimator is accordingly configured to estimate a localization error between the predicted bounding box and a ground-truth bounding box included in a labeled image, or a classification error between the classification label predicted by the learning model and a ground-truth label included in the labeled image.

It is to be understood that both the foregoing general description and the following detailed description are exemplary and explanatory only and are not restrictive of the disclosure, as claimed.

### BRIEF DESCRIPTION OF THE DRAWINGS

FIG. 1 illustrates three exemplary segmented images of a lung region.

FIG. 2 illustrates a schematic diagram of an exemplary image analysis system, according to certain embodiments of the present disclosure.

FIG. 3 illustrates a schematic diagram of a model training device, according to certain embodiments of the present disclosure.

FIG. 4A illustrates a schematic overview of a workflow performed by the model training device to train a main model and an error estimator using labeled images, according to certain embodiments of the present disclosure.

FIG. 4B illustrates a schematic overview of another workflow performed by the model training device to augment the training data by deploying the main model and the error estimator on unlabeled images, according to certain embodiments of the disclosure.

FIG. 5 illustrates a schematic overview of a training workflow performed by the model training device, according to certain embodiments of the present disclosure.

FIG. 6 is a flowchart of an example method for training a main model for performing an image analysis task along with an error estimator using labeled and unlabeled training data, according to certain embodiments of the disclosure.

FIG. 7A illustrates a schematic overview of a workflow performed by the model training device to train an image classification model and an error estimator using labeled images, according to certain embodiments of the present disclosure.

FIG. 7B illustrates a schematic overview of another workflow performed by the model training device to augment the training data by deploying the image classification model and the error estimator on unlabeled images, according to certain embodiments of the disclosure.

FIG. 8 is a flowchart of an example method for training an image classification model for performing an image classification task along with an error estimator using labeled and unlabeled training data, according to certain embodiments of the disclosure.

FIG. 9A illustrates a schematic overview of a workflow performed by the model training device to train an object detection model and an error estimator using labeled images, according to certain embodiments of the present disclosure.

FIG. 9B illustrates a schematic overview of another workflow performed by the model training device to augment the training data by deploying the object detection model and the error estimator on unlabeled images, according to certain embodiments of the disclosure.

FIG. 10 is a flowchart of an example method for training an object detection model for performing an object detection task along with an error estimator using labeled and unlabeled training data, according to certain embodiments of the disclosure.

FIG. 11A illustrates a schematic overview of a workflow performed by the model training device to train an image segmentation model and an error estimator using labeled images, according to certain embodiments of the present disclosure.

FIG. 11B illustrates a schematic overview of another workflow performed by the model training device to augment the training data by deploying the image segmentation model and the error estimator on unlabeled images, according to certain embodiments of the disclosure.

FIG. 12 is a flowchart of an example method for training an image segmentation model for performing an image segmentation task along with an error estimator using labeled and unlabeled training data, according to certain embodiments of the disclosure.

FIG. 13 is a flowchart of an example method for performing an image task on a medical image using a learning model trained with an error estimator, according to certain embodiments of the disclosure.

### DETAILED DESCRIPTION

Reference will now be made in detail to the exemplary embodiments, examples of hick are illustrated in the accompanying drawings.

The present disclosure provides an image analysis system and method for analyzing medical images acquired by an image acquisition device. The image analysis system and method that improve the training of learning models with low annotation cost using a novel error estimation model. The error estimation model automatically predicts the errors in the outputs of the current learning model on unlabeled samples and improves training by adding the unlabeled samples with low predicted error to the training dataset and requesting annotations for the unlabeled samples with high predicted error for guiding the learning model.

In some embodiments, training images used for training the learning model include a first set of labeled images and a second set of unlabeled images. The system and method first train the learning model and an error estimator with the first set of labeled images. The learning model is trained to perform an image analysis task and the error estimator is trained to estimate the error of the learning model associated with performing the image analysis task. The error estimator is then applied to the second set of unlabeled images to determine respective errors associated with the unlabeled images, and determine a third set of labeled images from the second set of unlabeled images based on the respective errors. An updated learning model is then trained with the first set of labeled images combined with the third set of labeled images.

The disclosed error estimation model aims to predict the difference between the main model's output and the underlying ground-truth, i.e., the error of the main model's prediction. It learns the error pattern of the main model and predicts the likely errors on even unseen unlabeled data. With the error estimation model, the disclosed system and method are thus able to select the unlabeled samples with likely low prediction error from the main learning model to add to the training dataset and augment training data, improving the training and leading to improved performance and generalization ability of the learning model. In some embodiments, they can also select the unlabeled samples with likely high prediction error to request human annotation, providing the most informative annotations for the main learning model. This leads to maximal use of limited human annotation resource. When the annotation task is dense (e.g., voxel-wise annotation for segmentation models), the image can be split into smaller patches or region of interests (ROI's) for sparse labeling.

Furthermore, the disclosed scheme allows an independent error estimator to be trained to learn the complex error patterns of arbitrary main model. This allows more flexibility and more thorough error estimation than some specific main model's limited built-in error estimation functionality which only captures certain type of errors under strict assumptions.

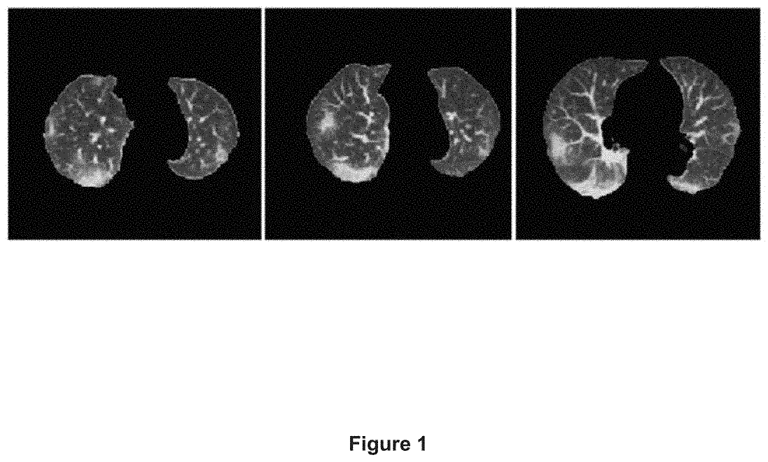

The disclosed system and method can be applied for any medical image analysis task (e.g., including classification, detection, segmentation, etc.) on any image modalities (e.g., including CT, X-ray, MRI, PET, ultrasound and others). Using segmentation task as an example, it is extremely time consuming to obtain voxel-level annotation for training purpose. For example, FIG. 1 illustrates three exemplary images of a lung region extracted from a 3D chest CT image. Each 2D image shown in FIG. 1 contains an annotated region of interest (ROI) of the lung region. The lung region shown in these images is confirmed to contract COVID-19 by positive RT-PCR test. As can be seen, the boundaries of the pneumonia regions are irregular and ambiguous, which makes detailed voxel-level delineation challenging even for experienced radiologists. Therefore, an improved training system and method for training learning models for medical image analysis with low annotation cost is needed.

Although FIG. 1 shows a medical image from a 3D chest CT scan, in some embodiments, the disclosed image analysis system may also perform image analysis on images acquired using other suitable imaging modalities, including, e.g., Magnetic Resonance Imaging (MRI), functional MRI (e.g., fMRI, DCE-MRI and diffusion MRI), Positron Emission Tomography (PET), Single-Photon Emission Computed Tomography (SPECT) X-ray, Optical Coherence Tomography (OCT), fluorescence imaging, ultrasound imaging, radiotherapy portal imaging, or the like. The present disclosure is not limited to any particular type of images.

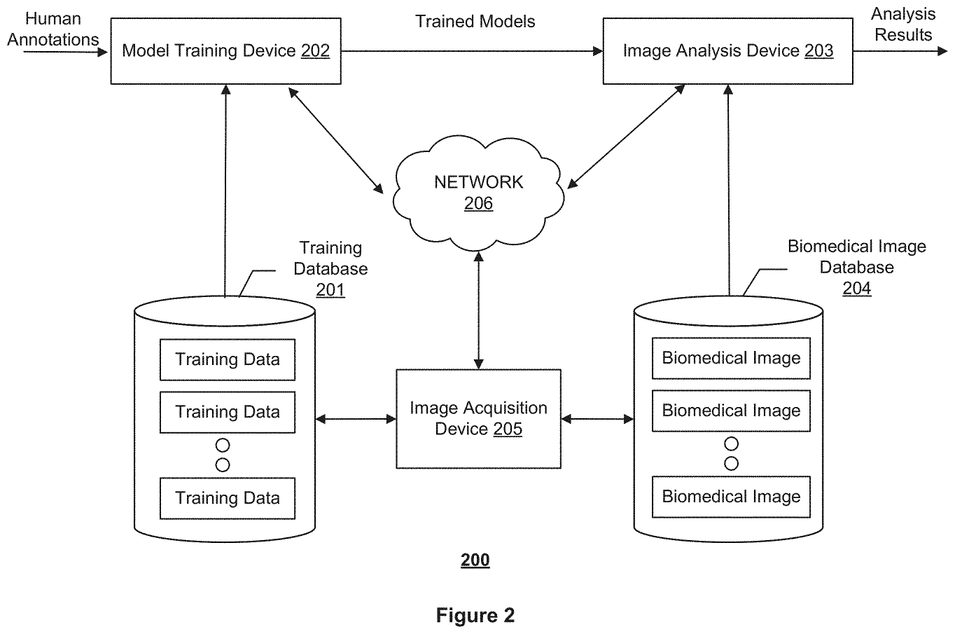

FIG. 2 illustrates an exemplary image analysis system 200, according to some embodiments of the present disclosure. As shown in FIG. 2, image analysis system 200 may include components for performing two phases, a training phase and a prediction phase. The prediction phase may also be referred to as an inference phase. To perform the training phase, image analysis system 200 may include a training database 201 and a model training device 202. To perform the prediction phase, image analysis system 200 may include an image analysis device 203 and a medical image database 204. In some embodiments, image analysis system 200 may include more or less of the components show) in FIG. 2.

Consistent with the present disclosure, image analysis system 200 may be configured to analyze a biomedical image acquired by an image acquisition device 205 and perform a diagnostic prediction based on the image analysis. In some embodiments, image acquisition device 205 may be a CT scanner that acquires 2D or 3D CT images. For example, image acquisition device 205 may be a 3D cone CT scanner for volumetric CT scans. In some embodiments, image acquisition device 205 may be using one or more other imaging modalities, including, e.g., Magnetic Resonance Imaging (MRI), functional MRI (e.g., fMRI, DCE-MRI and diffusion MRI), Positron Emission Tomography (PET), Single-Photon Emission Computed Tomography (SPECT), X-ray, Optical Coherence Tomography (OCT), fluorescence imaging, ultrasound imaging, radiotherapy portal imaging, or the like.

In some embodiments, image acquisition device 205 may capture medical images containing at east one anatomical structure or organ, such as a lung or a thorax. For example, each volumetric CT exam may contain 51˜1094 CT slices with a varying slice-thickness from 0.5 mm to 3 mm. The reconstruction matrix may have 512×512 pixels with in-plane pixel spatial resolution from 0.29×0.29 mm² to 0.98×0.98 mm².

In some embodiments, the acquired images may be sent to an annotation station 301 for annotating at least a subset of the images. In some embodiments, annotation station 301 may be operated by a user to provide human annotation. For example, the user may use keyboard, mouse, or other input interface of annotation station 301 to annotate the images, such as drawing boundary line of an object in the image, or identifying what anatomical structure the object is. In some embodiments, annotation station 301 may perform an automated or semi-automated annotation procedures to label the images. The labeled images may be included as part of training data provided to model training device 202.

Image analysis system 200 may optionally include a network 206 to facilitate the communication among the various components of image analysis system 200, such as databases 201 and 204, devices 202, 203, and 205. For example, network 206 may be a local area network (LAN), a wireless network, a cloud computing environment (e.g., software as a service, platform as a service, infrastructure as a service), a client-server, a wide area network (WAN), etc. In some embodiments, network 206 may be replaced by wired data communication systems or devices.

In some embodiments, the various components of image analysis system 200 may be remote from each other or in different locations and be connected through network 206 as shown in FIG. 2. In some alternative embodiments, certain components of image analysis system 200 may be located on the same site or inside one device. For example, training database 201 may be located on-site with or be part of model training device 202. As another example, model training device 202 and image analysis device 203 may be inside the same computer or processing device.

Model training device 202 may use the training data received from training database 201 to train a learning model (also referred to as a main learning model) for performing an image analysis task on a medical image received from, e.g., medical image database 204. As shown in FIG. 2, model training device 202 may communicate with training database 201 to receive one or more sets of training data, In some embodiments, training data may include a first subset of labeled data, e.g., labeled images, and a second subset of unlabeled data, e.g., unlabeled images. “Labeled data” is training data that includes ground-truth results obtained through human annotation and/or automated annotation procedures. For example, for an image segmentation task, the labeled data includes pairs of original images and the corresponding ground-truth segmentation masks for those images. As another example, for an image classification task, the labeled data includes pairs of original images and the corresponding ground-truth class labels for those ages. “Unlabeled data,” on the other hand, is training data that does not include the ground-truth results. Throughout the disclosure, labeled data/image may also be referred to as annotated data/image, and unlabeled data/image may also be referred to as unannotated data/image.

Consistent with the present disclosure, an error estimation model (also known as an error estimator) is trained along with the main learning model using the labeled data, to learn the error pattern of the main model. The trained error estimation model is then deployed to predict the likely errors on the unlabeled data. Based on this error prediction, unlabeled data with likely low prediction error may be annotated using the main learning model and then added to the labeled data to augment the training data. On the other hand, unlabeled data with likely high prediction error may be sent for human annotation and the manually labeled data is also added to the training data. The main learning model can then be trained using the augmented training data, thus improving performance and generalization ability of the learning model.

In some embodiments, the training phase may be performed “online” or “offline.” “Online” training refers to performing the training phase contemporarily with the prediction phase, e.g., learning the model in real-time just prior to analyzing a medical image. An “online” training may have the benefit to obtain a most updated learning model based on the training data that is then available. However, “online” training may be computational costive to perform and may not always be possible if the training data is large and/or the model is complicated, Consistent with the present disclosure, “offline” training is used where the training phase is performed separately from the prediction phase. The learned model trained offline is saved and reused for analyzing images.

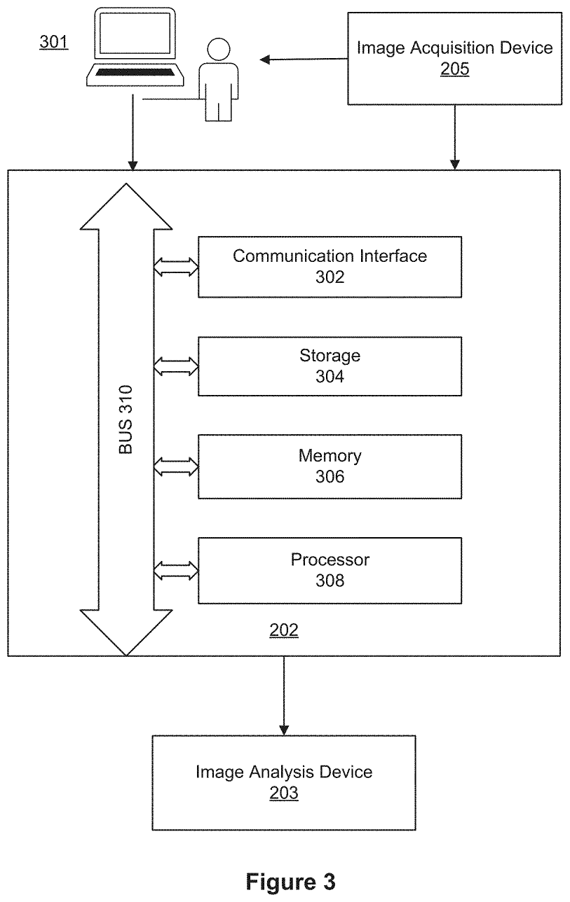

Model training device 202 may be implemented with hardware specially programmed by software that performs the training process. For example, model training device 202 may include a processor and a non-transitory computer-readable medium (discussed in detail in connection with FIG. 3). The processor may conduct the training by performing instructions of a training process stored in the computer-readable medium. Model training device 202 may additionally include input and output interfaces to communicate with training database 201, network 206, and/or a user interface (not shown). The user interface may be used for selecting sets of training data, adjusting one or more parameters of the training process, selecting or modifying a framework of the learning model, and/or manually or semi-automatically providing prediction results associated with an image for training.

Image analysis device 203 may communicate with medical image database 204 to receive medical images. The medical images may be acquired by image acquisition devices 205. Image analysis device 203 may automatically perform an image analysis task (e.g., segmentation, classification, object detection, etc.) on the medical images using the trained main learning model from model training device 202. Image analysis device 203 may include a processor and a non-transitory computer-readable medium (discussed in detail in connection with FIG. 3). The processor may perform instructions of a medical image diagnostic analysis program stored in the medium. Image analysis device 203 may additionally include input and output interfaces (discussed in detail in connection with FIG. 3) to communicate with medical image database 204, network 206, and/or a user interface (not shown). The user interface may be used for selecting medical images for analysis, initiating the analysis process, displaying the diagnostic results.

Systems and methods mentioned in the present disclosure may be implemented using a computer system, such as shown in FIG. 3. While FIG. 3 illustrates the detailed components inside model training device 202, it is contemplated that image analysis device 203 may include similar components, and the descriptions below with respect to the components of model training device 203 apply also to those of image analysis device 203, with or without adaption.

In some embodiments, model training device 202 may be a dedicated device or a general-purpose device. For example, model training device 202 may be a computer customized for a hospital to train learning models for processing image data. Model training device 202 may include one or more processor(s) 308 and one or more storage device(s) 304. The processor(s) 308 and the storage device(s) 304 may be configured in a centralized or distributed manner. Model training device 202 may also include a medical image database (optionally stored in storage device 304 or in a remote storage), an input/output device (not shown, but which may include a touch screen, keyboard, mouse, speakers/microphone, or the like), a network interface such as communication interface 302, a display (not shown, but which may be a cathode ray tube (CRT) or liquid crystal display (LCD) or the like), and other accessories or peripheral devices. The various elements of model training device 202 may be connected by a bus 310, which may be a physical and/or logical bus in a computing device or among computing devices.

The processor 308 may be a processing device that includes one or more general processing devices, such as a microprocessor, a central processing unit (CPU), a graphics processing unit (GPU), and the like. More specifically, the processor 308 may be a complex instruction set computing (CISC) microprocessor, a reduced instruction set computing (RISC) microprocessor, a very long instruction word (VLIW) microprocessor, a processor running other instruction sets, or a processor that runs a combination of instruction sets. The processor 308 may also be one or more dedicated processing devices such as application-specific integrated circuits (ASICs), field-programmable gate arrays (FPGAs), digital signal processors (DSPs), system-on-chip (SoCs), and the like.

The processor 308 may be communicatively coupled to the storage device 304 and configured to execute computer-executable instructions stored therein. For example, as illustrated in FIG. 3, a bus 310 may be used, although a logical or physical star or ring topology would be examples of other acceptable communication topologies. The storage device 304 may include a read-only memory (ROM), a flash memory, random access memory (RAM), a static memory, a volatile or non-volatile, magnetic, semiconductor, tape, optical, removable, nonremovable, or other types of storage device or tangible (e.g., non-transitory) computer-readable medium. In some embodiments, the storage device 304 may store computer-executable instructions of one or more processing programs and data generated when a computer program is executed, The processor may execute the processing program to implement each step of the methods described below. The processor may also send/receive image data to/from the storage device.

Model training device 202 may also include one or more digital and/or analog communication (input/output) devices, not illustrated in FIG. 3. For example, the input/output device may include a keyboard and a mouse or trackball that allow a user to provide input. Model training device 202 may further include a network interface, illustrated as communication interface 302, such as a network adapter, a cable connector, a serial connector, a USB connector, a parallel connector, a high-speed data transmission adapter such as optical fiber, USB 3.0, lightning, a wireless network adapter such as a WiFi adapter, or a telecommunication (3G, 4G/LTE, etc.) adapter and the like. Model training device 202 may be connected to a network through the network interface. Model training device 202 may further include a display, as mentioned above. In some embodiments, the display may be any display device suitable for displaying a medical image and its segmentation results. For example, the image display may be an LCD, a CRT, or an LED display.

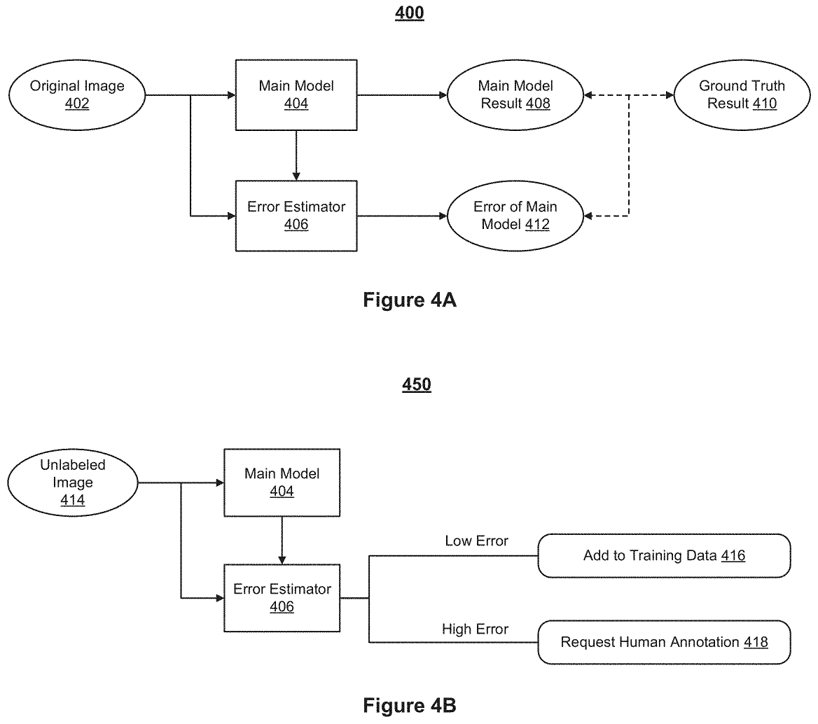

Model training device 202 may be connected to image analysis device 203 and image acquisition device 205 as discussed above with reference to FIG. 2. In some embodiments, model training device 202 may implement various workflows to train the learning model to be used by image analysis device 203 to perform a predetermined image analysis task, such as those illustrated in FIGS. 4A-4B, 5, 7A-7B, 9A-9B, and 11A-11B.

FIG. 4A illustrates a schematic overview of a workflow 400 performed by model training device to train a main model and an error estimator using labeled images, according to certain embodiments of the present disclosure. In workflow 400, labeled images are used as training samples to train a main model 404 and a separate error estimator 406. Each labeled image may include an original image 402 and a corresponding ground-truth result 410. Original image 402 may be a medical image acquired using any imaging modality, e.g., CT, X-ray, MRI, ultrasound, PET, etc. For example, original image 402 may be a medical image acquired by image acquisition device 205. In some embodiments, original image 402 may be pre-processed to improve image quality (e.g., to reduce noise, etc.) after being acquired by image acquisition device 205. Ground-truth result 410 may be an annotation of original image 402 depending on the image analysis task. For example, for classification tasks, ground-truth result 410 may be a binary or multi-class label indicating which class the input image belongs to. As another example, for object detection tasks, ground-truth result 410 can include the coordinates of bounding boxes of detected objects, and a class label for each object. As yet another example, for segmentation tasks, ground-truth results 410 can be an image segmentation mask with the same size as the input image indicating the class of each pixel in the input image, The annotation may be performed by a human (e.g., a physician or an image analysis operator) or by an automated process.

Original image 402 is input into main model 404. Main model 404 is a learning model configured to perform the main medical image analysis task (e.g., classification, object detection or segmentation). Main model 404 outputs a main model result 408 and the type of output is dependent on the image analysis task, similar to what is described above for ground-truth result 410. For example, for classification tasks, main model result 408 may be a class label; for object detection tasks, main model result 408 can be the coordinates of bounding boxes of detected objects, and a class label for each object; for segmentation tasks, main model result 408 can be an image segmentation mask. In some embodiments, the main model may be implemented by ResNet, U-Net, V-Net or other suitable learning models.

Error estimator may be another learning model configured to predict the errors in the main model's outputs, based on input image and the intermediate results of main model, such as the extracted feature maps. In some embodiments, error estimator 406 may receive original image 402 as an input. In some embodiments, error estimator 406 may additionally or alternatively receive certain intermediate results from main model 404, such as feature maps. Error estimator outputs an estimated error of main model 412. During training, error estimator 406 is trained by the error of main model 404, i.e., the difference between the main model result 408 and the ground-truth result 410 of the labeled data.

In some embodiments, the error estimator's training and inference are embedded as part of main model training. For example, in workflow 400, training of main model 404 and error estimator 406 may be performed sequentially or simultaneously. For example, each training sample may be used to train main model 404, and at the same time, the difference between the main model result 408 predicted using main model 404 and the ground-truth result 410 in the training sample is used to train and update error estimator. As another example, all the training samples in the training data may be used to train main model 404 first, and the differences between the main model results 408 and the ground-truth results 410 in the training samples may be collected used to train error estimator 406.

FIG. 4B illustrates a schematic overview of another workflow 450 performed by the model training device to augment the training data by deploying the main model and the error estimator on unlabeled images, according to certain embodiments of the disclosure. In workflow 450, error estimator 406 trained with workflow 400 is applied on unlabeled training data, e.g., unlabeled image 414, to predict errors yielded by main model 404. As shown, unlabeled image 414 and optionally certain intermediate results (e.g., features maps) from main model 404 when applied to the same unlabeled image 414, may be input to error estimator 406. Error estimator predicts an error of main model 404 using the input. If the predicted error is low, e.g., less than a predetermined threshold, unlabeled image 414 along with the main model result yielded by main model 404 is added to training data 416. Otherwise, if the predicted error is high, e.g., higher than a predetermined threshold, a human annotation 418 may be requested and the annotated image may be added to training data 416.

In some embodiments, to ensure error estimator 406 is performing at a good state and benefiting the training of main model 404, an optional independent labeled validation set may be used to validate the performance of error estimator 406. In some embodiments, the independent labeled validation set may be selected from the labeled training data and set aside for validation purpose. In order to keep it “independent,” the validation set will not be used as part of the labeled data to train main model 404 and error estimator 406. In one embodiment, the error estimator's performance can be evaluated through workflow 400, to directly compare the ground-truth error of main model 404 (e.g., the difference between ground-truth results 410 and the main model result 408) obtained on this validation set with the error estimation output by error estimator 406. In another embodiment, the error estimator's performance can be evaluated by evaluating the updated main model's performance on this validation set through workflow 450, using the low-error and high-error data identified by error estimator 406, and compare it against the initial main model's performance with only labeled data on the validation set. These validations provide extra assurance that the error estimator is performing well and providing benefits for training main model.

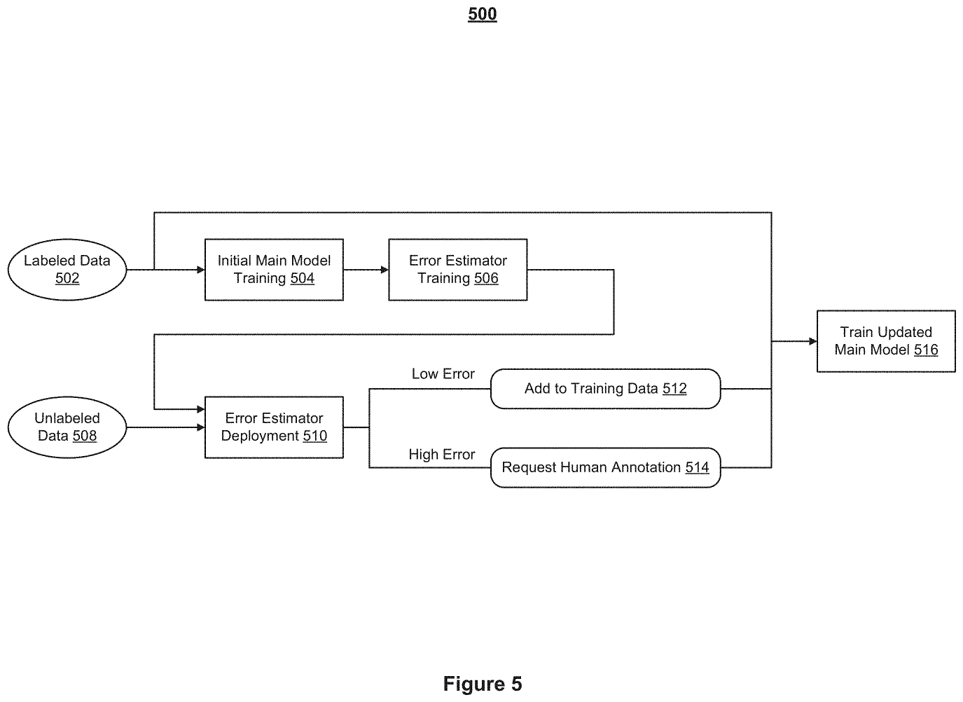

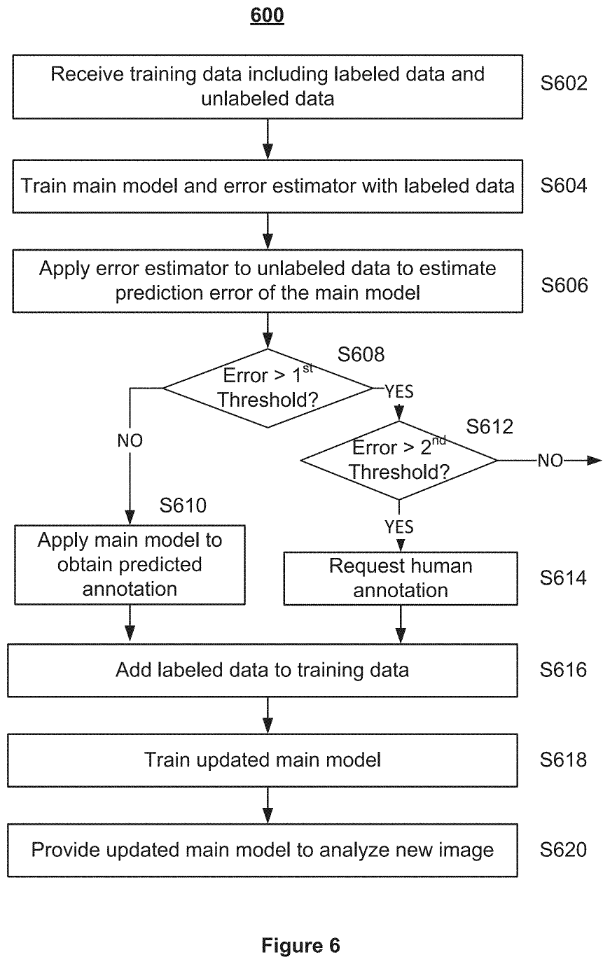

FIG. 5 illustrates a schematic overview of a training workflow 500 performed by the model training device, according to certain embodiments of the present disclosure. FIG. 6 is a flowchart of an example method 600 for training a main model for performing an image analysis task along with an error estimator using labeled and unlabeled training data, according to certain embodiments of the disclosure, Method 600 may be performed by model training device 202 and may include steps S602-S620. It is contemplated that some steps may be optional and certain steps may be performed in an order different from shown in FIG. 6. FIGS. 5-6 will be described together.

Method 600 starts when model training device 202 receives training data (step S602). For example, training data may be received from training database 201. In some embodiments, the training data includes a first subset of labeled data (e.g., labeled data 502 in workflow 500) and a second subset of unlabeled data (e.g., unlabeled data 508 in workflow 500). For example, training data may include labeled and unlabeled images. In some embodiments, the training images may be acquired using the same imaging modality as those will later be analyzed by the main model, to enhance the training accuracy. The imaging modality may be any suitable one, including, e.g., MRI, fMRI, DCE-MRI, diffusion MRI, PET, SPECT, X-ray, OCT, fluorescence imaging, ultrasound imaging, radiotherapy portal imaging, or the like,

Model training device 202 then trains an initial main model and an error estimator with the labeled data (step S604). The main model is trained to take input image and predict an output of the designated image analysis task (segmentation/classification/detection, etc.). The error estimator can take original input image or main model's intermediate result or feature maps as input. For example, as shown in workflow 500, initial main model training 504 and error estimator training 506 are performed using labeled data 502. In some embodiments, initial main model training 504 uses the ground-truth results included in labeled data 502, while error estimator training 506 relies on the difference between the ground-truth results and the predicted results using initial main model.

Model training device 202 then applies the error estimator trained in step S604 to estimate the prediction error of the main model (step S606). For example, as shown in workflow 500, error estimator deployment 510 is performed by applying the error estimator provided by error estimator training 506 on unlabeled data 508 to estimate the prediction error of the main model provided by initial main model training 504.

Model training device 202 determines whether the estimated error exceeds a predetermined first threshold (step S608). In some embodiments, the first threshold may be a relatively low value, e.g., 0.1. If the error does not exceed the first threshold (S608: No), the error is considered low, and model training device applies the initial main model to obtain a predicted annotation of the unlabeled data (step S610) to form a labeled data sample and the labeled data sample is added to the training data (step S614). For example, in workflow 500, when the error is likely “low,” the unlabeled data 508 along with the prediction result by the trained initial main model (the “pseudo-annotation”) is added to training data 512. These samples can augment training data and improve the performance and generalization ability of main model.

Otherwise, if the error exceeds the first threshold (S608: Yes), model training device 202 further determines whether the estimated error exceeds a predetermined second threshold (step S612). In some embodiments, the second threshold may be a relatively high value, higher than the first threshold, e.g., 0.9. If the error exceeds the second threshold (S612: Yes), the error is considered high, and model training device 202 requests a human annotation on the unlabeled data (step S614) to form a labeled data sample and the manually labeled data sample is added to the training data (step S616). For example, in workflow 500, when the error is likely “high,” human annotation 514 is requested, and the unlabeled data 508 along with the human annotation 514 is added to training data 512. These human annotated samples are most informative for improving the main model as the initial main model is expected to perform poorly on them, according to the error estimator. Accordingly, the limited annotation resource is leveraged to achieve optimal performance in annotation efficient learning scenarios. The training data is thus augmented by including the automatically (by the main model) or manually (by human annotation) labeled data.

Using the augmented training data, model training device 202 trains an updated main model (step S618) to replace the initial main model trained using just the labeled data included in the initial training data. For example, in workflow 500, three sources of labeled data are used to train updated main model 516: the originally labeled data 502, the low-error portion of unlabeled data 508 with initial main model outputs as pseudo-annotations, and the high-error portion of unlabeled data 508 with newly requested human annotations.

In some embodiments, due to the limited human annotation resource, not all high-error unlabeled data can be annotated by human in step S614. In this case, the second threshold can be selected high, so that model training device 202 can request the data with highest predicted error according to error estimator to be annotated first, in step S614. In some embodiments, some data may remain unlabeled, neither pseudo-labeled by main model nor manually labeled by request. For example, if the error exceeds the first threshold (S608: Yes) but does not exceed the second threshold (S612: No), the data sample may remain unlabeled during this iteration of update. Workflow 500 shown in FIG. 5 can be repeated, once or multiple times, to use the updated main model (trained in step S618) as the initial main model, and update it again. As the main model becomes stronger, there may be more data that can be pseudo-labeled by the main model and the unlabeled portion of the data will be further reduced.

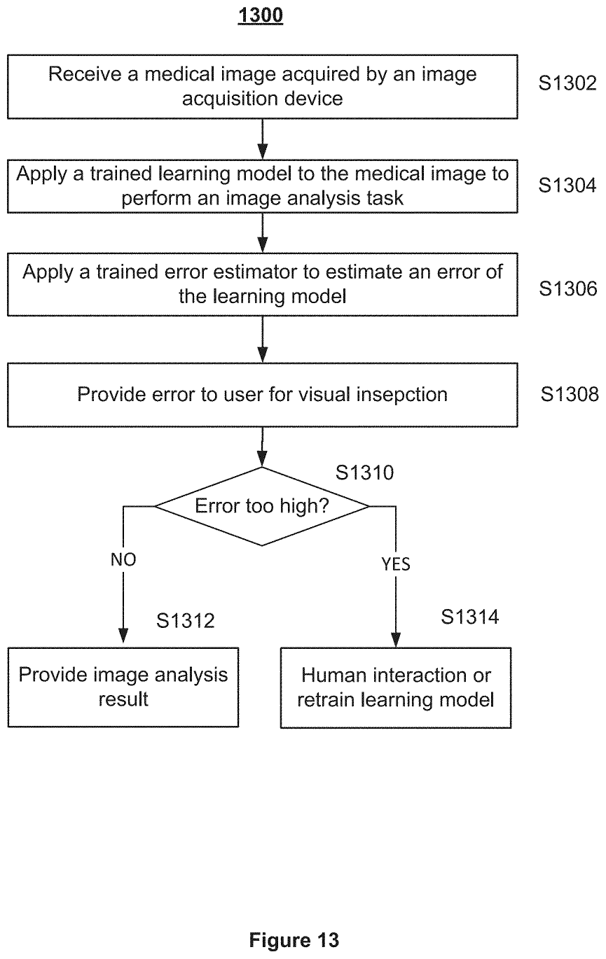

Model training device 202 then provides the updated main model as the learning model for analyzing new medical images (step S620). The training method 600 then concludes. The updated main model can be deployed, by image analysis device 203, to accomplish the designated medical image analysis task on new medical images. In some embodiments, the error estimator can be disabled if error estimation of the main model is not desired in the application. In some alternative embodiments, the error estimator can be kept on to provide estimation of potential error in the main model's output. For example, the error estimator can be used to generate an error of the main model in parallel to the main model performing an image analysis task, and provide that error to user for visual inspection, e.g., through a display of image analysis device 203, such that the user understands the performance of the main model. More details related to applying the trained model and error estimator will be provided in connection FIG. 13 below.

By identifying unlabeled data that will cause a high prediction error when applying the main model, and only requesting human annotation on such unlabeled data, method 600 can allocate limited human annotation resources to analyze only the images that cannot be accurately analyzed by the main model. By including the automatically and manually annotated data (e.g., the pseudo-annotations and human annotations) to augment the training data, method 600 also helps the main model training to make the best of existing unlabeled data.

The main model may be trained to perform any predetermined image analysis task, e.g., image segmentation, image classification, and object detection from the image, etc. Based on the specific image analysis task, the features extracted by the main model during prediction, the prediction results, the ground-truth results included in the labeled data, the error estimated by the error estimator, the configuration of the learning model and the configuration of the error estimator, may all be designed accordingly.

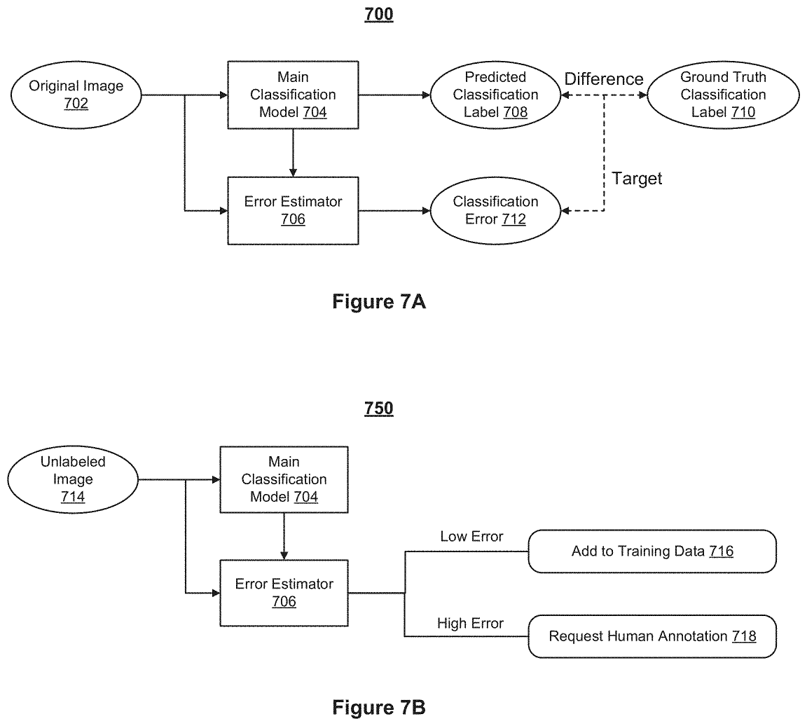

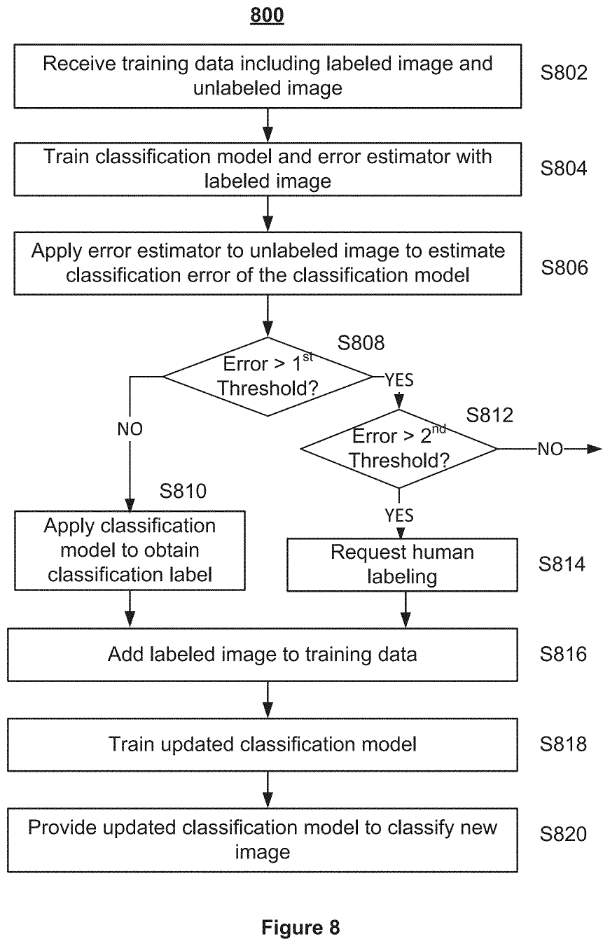

For example, when the image analysis task is image classification, the main model may be an image classification model configured to predict a class label for the image. In this case, the output of main model is a binary or multi-class classification label. The output of error estimator is a classification error, e.g., a cross entropy loss between the prediction and ground-truth label. FIG. 7A illustrates a schematic overview of a workflow 700 performed by model training device 202 to train a main classification model 704 and an error estimator 706 using labeled images, according to certain embodiments of the present disclosure. FIG. 7B illustrates a schematic overview of another workflow 750 performed by the model training device to augment the training data by deploying main classification model 704 and error estimator 706 on unlabeled images, according to certain embodiments of the disclosure. FIG. 8 is a flowchart of an example method 800 for training an image classification model for performing an image classification task along with an error estimator using labeled and unlabeled training data, according to certain embodiments of the disclosure. Method 800 may be performed by model training device 202 and may include steps S802-S820. It is contemplated that some steps may be optional and certain steps may be performed in an order different from shown in FIG. 8. FIGS. 7A-7B and 8 will be described together.

Method 800 starts when model training device 202 receives training data (step S802) similar to step S602 described above. Model training device 202 then trains a main classification model and an error estimator with the labeled data (step S804). As shown in workflow 700, main classification model 704 is trained to take original image 702 as input and predict a classification label as the output. Error estimator 706 can take original image 702 or main model's intermediate results or feature maps as input. As shown in FIG. 7A, main classification model 704 and error estimator 706 are initially trained using labeled data including the pairs of the original image 702 and its corresponding ground-truth classification label 710. In some embodiments, main classification model 704 is trained to minimize the difference between a predicted classification label 708 when applying main classification model 704 to original image 702 and ground-truth classification label 710 corresponding to original image 702. In some embodiments, main classification model 704 may be implemented by any classification network, including ResNet, EfficientNet, NAS, etc.

Error estimator 706, on the other hand, is trained using a “ground-truth error” determined using ground-truth classification label 710 and predicted classification label 708. In one example, the error may be a cross entropy loss between ground-truth classification label 710 and predicted classification label 708. Training of error estimator 706 aims to minimize the difference between an estimated classification error 712 estimated by error estimator 706 and the “ground-truth error” determined using ground-truth classification label 710 and predicted classification label 708. In some embodiments, error estimator 706 may be implemented by a multi-layer perceptron or other networks.

Model training device 202 then applies the error estimator trained in step S804 to estimate the classification error of the main classification model (step S806). For example, as shown in workflow 750, error estimator 706 is applied on unlabeled image 714 to estimate the classification error of main classification model 704.

Model training device 202 determines whether the estimated classification error exceeds a predetermined first threshold (step S808). In some embodiments, the first threshold can be a low value, e.g., 0.1. If the classification error does not exceed the threshold (S808: No), model training device 202 applies main classification model 704 to obtain a predicted classification label of the unlabeled data (step S810) to form a pseudo-labeled data sample and the pseudo-labeled data sample is added to the training data (step S816). For example, in workflow 700, when the classification error is likely “low,” the unlabeled image 714 along with the classification label predicted by the main classification model 704 is added to training data 716.

Otherwise, if the classification error exceeds the first threshold (S808: Yes), model training device 202 determines whether the estimated classification error exceeds a predetermined second threshold (step S812). In some embodiments, the second threshold can be a high value higher than the first threshold, e.g., 0.9. If the classification error exceeds the second threshold (S812: Yes), model training device 202 requests a human annotation on the unlabeled image (step S814) to form a manually labeled data sample, which is then added to the training data (step S816). For example, in workflow 750, when the classification error is likely “high,” human annotation 718 is requested, and the unlabeled image 714 along with the human annotation 718 is added to training data 716. If the error exceeds the first threshold (S808: Yes) but does not exceed the second threshold (S812: No), the data sample may remain unlabeled.

Using the augmented training data, model training device 202 trains an updated main classification model (step S818) to replace the initial main classification model trained using just the labeled images, and provides the updated main classification model as the learning model for analyzing new medical images (step S820), similar to steps S618 and S620 described above in connection with FIG. 6. The updated main classification model can be deployed to predict a binary or multi-class label for new medical images.

As another example, when the image analysis task is object detection, the main model may be an object detection model (also referred to as a detector model) configured to detect an object. In this case, the output of main model includes coordinates of a bounding box surrounding the object and a class label for the object. The output of error estimator includes a localization error, e.g., the mean square difference between the predicted and ground-truth bounding box coordinates, and/or a classification error, e.g., the cross-entropy loss between predicted and ground-truth object class labels.

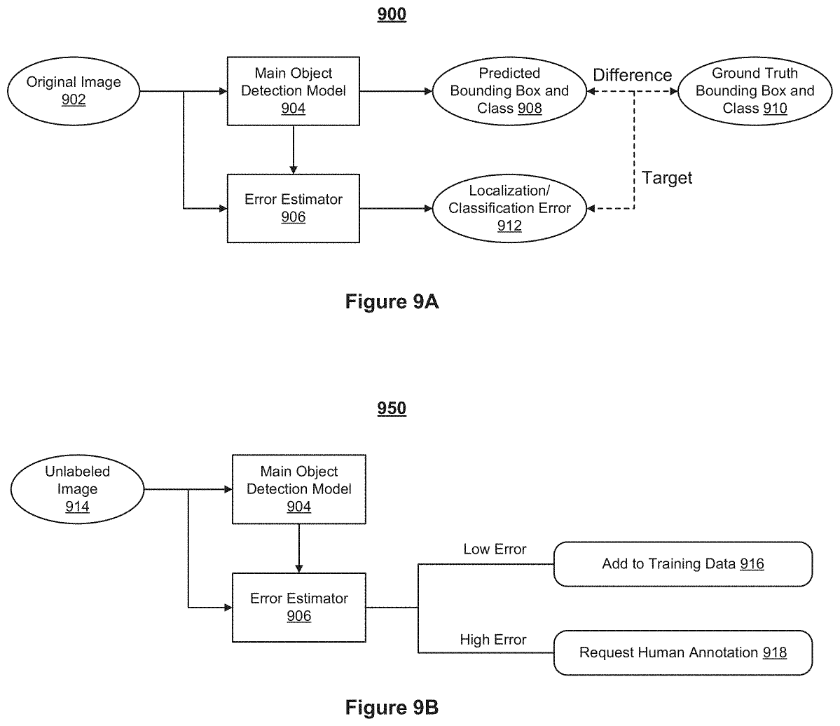

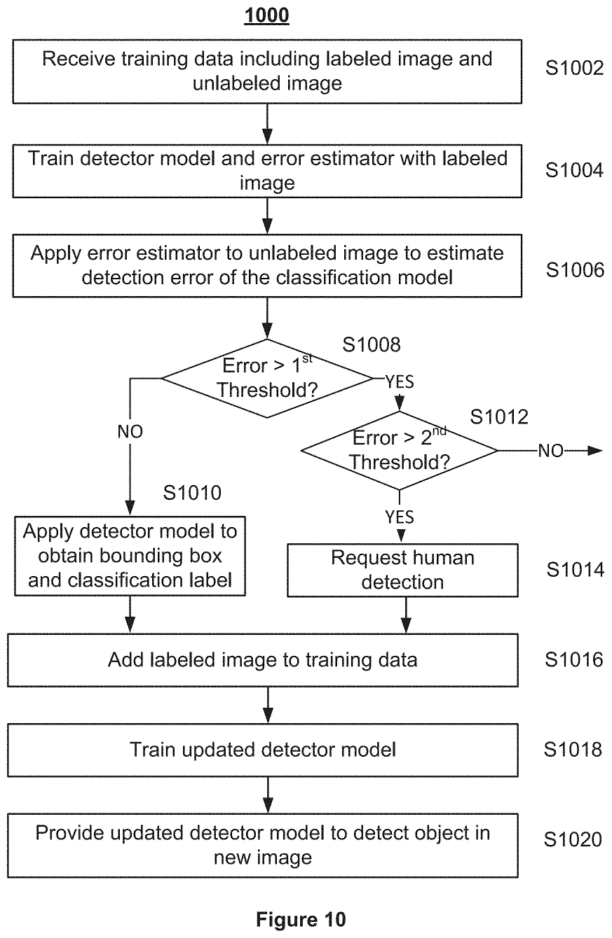

FIG. 9A illustrates a schematic overview of a workflow 900 performed by model training device 202 to train an object detection model 904 and an error estimator 906 using labeled images, according to certain embodiments of the present disclosure. FIG. 9B illustrates a schematic overview of another workflow 950 performed by the model training device to augment the training data by deploying object detection model 904 and error estimator 906 on unlabeled images, according to certain embodiments of the disclosure. FIG. 10 is a flowchart of an example method 1000 for training an object detection model for performing an object detection task along with an error estimator using labeled and unlabeled training data, according to certain embodiments of the disclosure. Method 1000 may be performed by model training device 202 and may include steps S1002-S1020. It is contemplated that some steps may be optional and certain steps may be performed in an order different from shown in FIG. 10. FIGS. 9A-9B and 10 will be described together.

Method 1000 starts when model training device 202 receives the training data (step S1002) similar to step S802 described above. Model training device 202 then trains a main object detection model and an error estimator with the labeled data (step S1004). As shown in workflow 900, main object detection model 904 is trained to take original image 902 as input and predict coordinates of an object bounding box and a class label of the object as the outputs. Error estimator 906 can take original image 902 or main model's intermediate results or feature maps as input. As shown in 9A, main object detection model 904 and error estimator 906 are initially trained using labeled data including the pairs of the original image 902 and its corresponding ground-truth bounding box and classification label 910. In some embodiments, main object detection model 904 is trained to minimize the difference between the predicted and ground-truth bounding boxes and classes. In some embodiments, main object detection model 904 may be implemented by any object detection network, including R-CNN, YOLO, SSD, CenterNet, CornerNet, etc.

Error estimator 906, on the other hand, is trained using a “ground-truth error” determined using ground-truth bounding box and classification label 910 and predicted bounding box and classification label 908. In one example, the error may be a cross entropy loss between ground-truth classification label 910 and predicted classification label 908. Training of error estimator 906 aims to minimize the difference between an estimated localization and/or classification error 912 estimated by error estimator 906 and the “ground-truth error.” In some embodiments, error estimator 906 may be implemented by two multi-layer perceptions, for estimating localization and classification errors respectively, or other types of networks.

Model training device 202 then applies the error estimator trained in step S1004 to estimate the localization error and/or classification error of the main object detection model (step S1006). For example, as shown in workflow 950, error estimator 906 is applied on unlabeled image 914 to estimate the localization error and/or classification error of main object detection model 904, In some embodiments, error estimator 906 may further determine a combined error reflecting both localization and classification errors, e.g., as a weighted sum of the two errors, or otherwise aggregating the two errors.

Steps S1008-S1020 are performed similar to steps S808-S820 above in connection with FIG. 8 except the annotation in this scenario includes the bounding box and class label of the detected object. Detailed descriptions are not repeated.

As yet another example, when the image analysis task is image segmentation, the main model may be a segmentation model configured to segment an image. In this case, the output of main model is a segmentation mask. The output of the error estimator is an error map of the segmentation mask. If the image to be segmented is 3D image, the segmentation mask is accordingly a voxel-wise segmentation mask, the error map is a voxel-wise map, e.g., a voxel-wise cross entropy loss map.

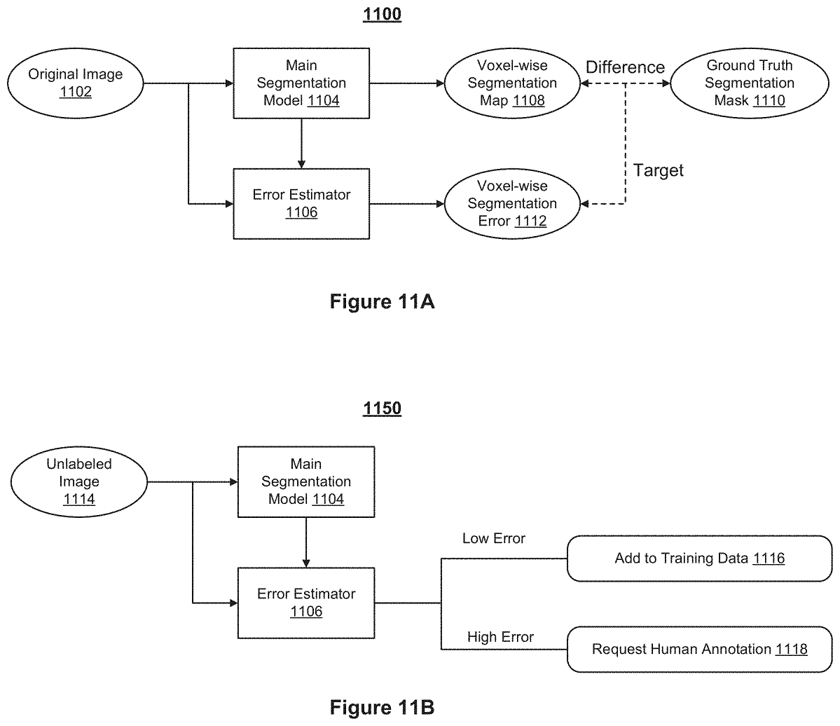

FIG. 11A illustrates a schematic overview of a workflow 1100 performed by model training device 202 to train a main segmentation model 1104 and an error estimator 1106 using labeled images, according to certain embodiments of the present disclosure. FIG. 11B illustrates a schematic overview of another workflow 1150 performed by the model training device to augment the training data by deploying main segmentation model 1104 and error estimator 1106 on unlabeled images, according to certain embodiments of the disclosure.

Workflows 1100/1150 are similar workflows 700/750 and workflows 900/950 described above in connection with FIGS. 7A-7B and 9A-9B, except prediction result of main segmentation model 1104, when applied to original image 1102, is a segmentation mask 1108 and the error estimated by error estimator 1106 is a segmentation error map 1112. A ground-truth segmentation mask 1110 corresponding to original image 1102 included in the labeled image is used to train main segmentation model 1104, as well as to determine the “ground-truth” segmentation error map used to train error estimator 1106. In some embodiments, the segmentation error map may be a voxel-wise cross entropy loss map. Detailed descriptions of workflows 1100/1150 can be found and adaptive from those of workflows 700/750 and workflows 900/950 described above, and therefore are not repeated,

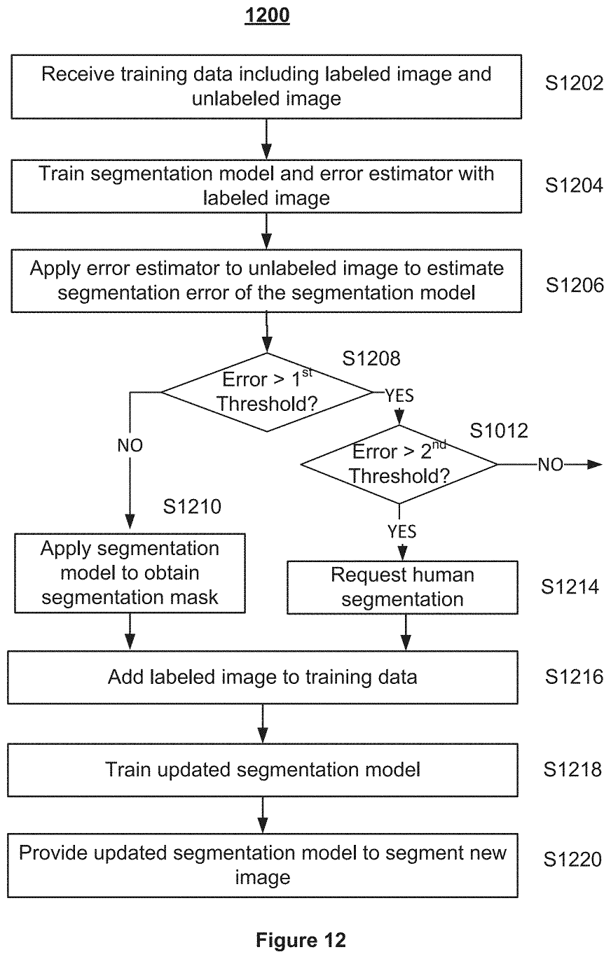

FIG. 12 is a flowchart of an example method 1200 for training a segmentation model for performing an image segmentation task along with an error estimator using labeled and unlabeled training data, according to certain embodiments of the disclosure. Method 1200 may be performed by model training device 202 and may include steps S1202-S1220. It is contemplated that some steps may be optional and certain steps may be performed in an order different from shown in FIG. 12.

Method 1200 starts when model training device 202 receives the training data (step S1202) similar to steps S802 and S1002 described above. Model training device 202 then trains a main segmentation model and an error estimator with the labeled data (step S1204). As shown in workflow 1100, main segmentation model 1104 is trained to take original image 1102 as input and predict a segmentation mask as the output. Error estimator 1106 can take original image 1102 or main model's intermediate results or feature maps as input. As shown in FIG. 11A, main segmentation model 1104 and error estimator 1106 are initially trained using labeled data including the pairs of the original image 1102 and its corresponding ground-truth segmentation mask 1110. In some embodiments, main segmentation model 1104 is trained to minimize the difference between the predicted and ground-truth bounding boxes and classes. In some embodiments, main segmentation model 1104 may be implemented by any segmentation network, including U-Net, V-Net, DeepLab, Feature Pyramid Network, etc.

Error estimator 1106, on the other hand, is trained using a “ground-truth error” determined using ground-truth segmentation mask 1110 and predicted segmentation mask 1108. In one example, the error may be a cross entropy loss map determined based on ground-truth segmentation mask 1110 and predicted segmentation mask 1108. Training of error estimator 1106 aims to minimize the difference between an estimated segmentation error map 1112 estimated by error estimator 1106 and the “ground-truth error.” Error estimator 1106 may be implemented by a decoder network in U-Net or other types of segmentation networks.

Model training device 202 then applies the error estimator trained in step S1204 to estimate the segmentation error map of the main segmentation model (step S1206). For example, as shown in workflow 1150, error estimator 1106 is applied on unlabeled image 1114 to estimate the segmentation error map of main segmentation model 1104.

Steps S1208-S1220 are performed similar to steps S808-S820 above in connection with FIG. 8 and steps S1008-S1020 above in connection with FIG. 10 except the annotation in this scenario is a segmentation mask. Detailed descriptions are not repeated.

Due to the dense nature of the image segmentation task, annotating the whole image can be expensive. The main segmentation model may only make mistakes at certain regions of the image, In some embodiments, to further improve annotation efficiency, images can be broken into patches or ROIs (region of interests) after they are received in step S1202 and before training is performed in step S1204. Accordingly, steps S1206-S1218 can be performed on a patch/ROI basis. For example, the main segmentation model can predict the segmentation mask for each patch or ROI, and the error estimator can assess errors in each patch or ROI instead of whole image to provide finer-scale guidance. In another example, the main segmentation model and error estimator can predict the segmentation mask and error estimation for the whole image, but only patches or ROIs containing large amount of error as indicated by the error estimator are provided to annotator for further annotation. In such embodiments, the annotator may be prompted to only annotate in a smaller region where the main model is likely wrong in step S1214, greatly alleviating annotation burden. The annotation could be manually, semi-manually or fully automatically obtained. For example, a more expensive model/method could be used to automatically generate the annotation. The annotation could also obtain, semi-automatically or automatically, with the aid of other imaging modalities.

FIG. 13 is a flowchart of an example method 1300 for performing an image task on a medical image using a learning model trained with an error estimator, according to certain embodiments of the disclosure. Method 1300 may be performed by image analysis device 203 and may include steps S1302-S1314. It is contemplated that some steps may be optional and certain steps may be performed in an order different from shown in FIG. 13.

Method 1300 starts when image analysis device 203 receives a medical image acquired by an image acquisition device (step S1302). In some embodiments, image analysis device 203 may receive the medical image directly from image acquisition device 205, or from medical image database 204, where the acquired images are stored. Again, the medical image can be acquired using any imaging modality, including, e.g., CT, Cone-beam CT, MRI, fMRI, DCE-MRI, diffusion MRI, PET, SPECT, X-ray, OCT, fluorescence imaging, ultrasound imaging, radiotherapy portal imaging, or the like.

Image analysis device 203 then applies a trained learning model to the medical image to perform an image analysis task (step S1304). In some embodiments, the learning model may be jointly trained with a separate error estimator on partially labeled training images. For example, the learning model may be updated main model 516 trained using workflow 500 of FIG. 5 or method 600 of FIG. 6.

In steps S1304 and S1306, the image analysis task may be any predetermined task to analyze or otherwise process the medical image. In some embodiments, the image analysis task is an image segmentation task, and the learning model is designed to predict a segmentation mask of the medical image, e.g., a segmentation mask for a lesion in the lung region. The segmentation mask can be a probability map. For example, the segmentation learning model and error estimator can be trained using workflow 1100/1150 of FIG. 11A-11B and method 1200 of FIG. 12. In some embodiments, the image analysis task is an image classification task, the learning model is designed to predict a classification label of the medical image. For example, the classification label may be a binary label to indicate whether the medical image contains a tumor, or a multi-class label that indicate what type of tumor the medical image contains. For example, the classification learning model and error estimator can be trained using workflow 700/750 of FIG. 7A-7B and method 800 of FIG. 8. In some embodiments, the image analysis task is an object detection task, the learning model is designed to detect an object from the medical image, e.g., by predicting a bounding box surrounding the object and a classification label of the object. For example, coordinates of the bounding box of a lung nodule can be predicted and a class label can be predicted to indicate it is a lung nodule. For example, the object detection learning model and error estimator can be trained using workflow 900/950 of FIG. 9A-9B and method 1000 of FIG. 10.

Image analysis device 203 may also apply the trained error estimator to the medical image to estimate an error of the learning model when performing the image analysis task on the medical image (step S1306). In some embodiments, the error estimator can be applied to generate the error in parallel to the main model performing the image analysis task in step S1304. The type of error estimated by error estimator depends on the image analysis task. For example, when the image analysis task is image segmentation, the error estimator can be designed to estimate an error map or error estimation of the segmentation mask. When the image analysis task is image classification, the error estimator is accordingly designed to estimate a classification error, such as a cross entropy loss, between the classification label predicted by the learning model and a ground-truth label included in a labeled image. When the image analysis task is object detection, the error estimator is accordingly configured to estimate a localization error between the predicted bounding box and a ground-truth bounding box included in a labeled image, or a classification error between the classification label predicted by the learning model and a ground-truth label included in the labeled image, or the combination of the two.

Image analysis device 203 may provide the error estimated in step S1306 to a user for visual inspection (step S1308). For example, the error can be an error map provided as an image through a display of image analysis device 203, such that the user understands the performance of the main model,

In step S1310, it is determined whether the error is too high. In some embodiments, the determination can be made by the user as a result of the visual inspection. In some alternative embodiments, the determination can be made automatically by image analysis device 203 by, e.g., by comparing the error to a threshold. If the error is too high (S1310: Yes), image analysis device 203 may request user interaction to improve the learning model or request the learning model to be retrained by model training device 202 (step S1314). Image analysis device 203 repeat steps S1306-S1310 with the user-improved or retained new learning model. For example, the learning model may be updated using workflow 500 of FIG. 5, using the current learning model as the initial main model. Otherwise (S1310: No), image analysis device 203 may provide the image analysis results (step S1312), such as the classification label, the segmentation mask, or the bounding boxes.

According to certain embodiments, a non-transitory computer-readable medium may have a computer program stored thereon. The computer program, when executed by at least one processor, may perform a method for biomedical image analysis. For example, any of the above-described methods may be performed in this way.

In some embodiments, the computer-readable medium may include volatile or non-volatile, magnetic, semiconductor, tape, optical, removable, non-removable, or other types of computer-readable medium or computer-readable storage devices. For example, the computer-readable medium may be the storage device or the memory module having the computer instructions stored thereon, as disclosed, In some embodiments, the computer-readable medium may be a disc or a flash drive having the computer instructions stored thereon.

It will be apparent to those skilled in the art that various modifications and variations can be made to the disclosed system and related methods. Other embodiments will be apparent to those skilled in the art from consideration of the specification and practice of the disclosed system and related methods.

It is intended that the specification and examples be considered as exemplary only, with a true scope being indicated by the following claims and their equivalents.

What is claimed is:

1. A system for analyzing medical images using a learning model, comprising:
a communication interface configured to receive a medical image acquired by an image acquisition device; and
at least one processor, configured to apply the learning model to perform an image analysis task on the medical image,
wherein the learning model is trained jointly with an error estimator using training images comprising a first set of labeled images and a second set of unlabeled images, wherein the error estimator is configured to estimate an error of the learning model associated with performing the image analysis task.

2. The system of claim 1, wherein the at least one processor is further configured to:
apply the error estimator to the medical image to estimate the error of the learning model when performing the image analysis task on the medical image.

3. The system of claim 2, further comprising a display configured to provide the error to a user for visual inspection.

4. The system of claim 1, wherein to train the learning model and the error estimator, the at least one processor is configured to:
train an initial version of the learning model and an error estimator with the first set of labeled images;
apply the error estimator to the second set of unlabeled images to determine respective errors associated with the unlabeled images;
determine a third set of labeled images from the second set of unlabeled images based on the respective errors; and
train an updated version of the learning model with the first set of labeled images combined with the third set of labeled images; and
provide the updated version of the learning model to perform the image analysis task on the medical images.

5. The system of claim 4, wherein, to determine the third set of labeled images from the second set of unlabeled images, the at least one processor is further configured to:
identify at least one unlabeled image from the second set of unlabeled images associated with an error lower than a predetermined first threshold;
apply the learning model to the identified unlabeled image to generate a corresponding pseudo-labeled image; and
include the pseudo-labeled image into the third set of labeled images,

6. The system of claim 4, wherein, to determine the third set of labeled images from the second set of unlabeled images, the at least one processor is further configured to:
identify at least one unlabeled image from the second set of unlabeled images associated with an error higher than a predetermined second predetermined threshold;
obtain an annotation on the identified unlabeled image to form a corresponding new labeled image; and
include the new labeled image into the third set of labeled images.

7. The system of claim 4, wherein the first set of labeled images comprise original images and corresponding ground-truth results,
wherein the error estimator is trained based on differences between the ground-truth results in the first set of labeled images and image analysis results obtained by applying the learning model to the original images in the first set of labeled images.

8. The system of claim 1, wherein the image analysis task is an image segmentation task, and the learning model is configured to predict a segmentation mask, wherein the error estimator is configured to estimate an error map of the segmentation mask.

9. The system of claim 1, wherein the image analysis task is an image classification task, the learning model is configured to predict a classification label,
wherein the error estimator is configured to estimate a classification error between the classification label predicted by the learning model and a ground-truth label included in a labeled image.

10. The system of claim 1, wherein the image analysis task is an object detection task, the learning model is configured to predict a bounding box surrounding an object and a classification label of the object.

11. The system of claim 10, wherein the error estimator is configured to estimate a localization error between the predicted bounding box and a ground-truth bounding box included in a labeled image, or a classification error between the classification label predicted by the learning model and a ground-truth label included in the labeled image.

12. A computer-implemented method for analyzing medical images using a learning model, comprising:
receiving, by a communication interface, a medical image acquired by an image acquisition device; and
applying, by at least one processor, the learning model to perform an image analysis task on the medical image,
wherein the learning model is trained jointly with an error estimator using training images comprising a first set of labeled images and a second set of unlabeled images, wherein the error estimator is configured to estimate an error of the learning model associated with performing the image analysis task.

13. The computer-implemented method of claim 12, further comprising:
applying the error estimator to the medical image to estimate the error of the learning model when performing the image analysis task on the medical image; and
providing the error to a user via a display for visual inspection.

14. The computer-implemented method of claim 12, where the learning model and the error estimator are trained by:
training an initial version of the learning model and an error estimator with the first set of labeled images;
applying the error estimator to the second set of unlabeled images to determine respective errors associated with the unlabeled images;
determining a third set of labeled images from the second set of unlabeled images based on the respective errors;
training an updated version of the learning model with the first set of labeled images combined with the third set of labeled images; and
providing the updated version of the learning model to perform the image analysis task on the medical images.

15. The computer-implemented method of claim 14, wherein determining the third set of labeled images from the second set of unlabeled images further comprises:
identifying at least one unlabeled image from the second set of unlabeled images associated with an error lower than a predetermined first threshold;
applying the learning model to the identified unlabeled image to generate a corresponding pseudo-labeled image; and
including the pseudo-labeled image into the third set of labeled images.

16. The computer-implemented method of claim 14, wherein determining the third set of labeled images from the second set of unlabeled images further comprises:
identifying at least one unlabeled image from the second set of unlabeled images associated with an error higher than a predetermined second threshold;
obtaining a human annotation on the identified unlabeled image to form a corresponding new labeled image; and
including the new labeled image into the third set of labeled images.

17. The computer-implemented method of claim 12, wherein the image analysis task is an image segmentation task, and the learning model is configured to predict a segmentation mask,
wherein the error estimator is configured to estimate an error map of the segmentation mask.

18. The computer-implemented method of claim 12, wherein the image analysis task is an image classification task, the learning model is configured to predict a classification label,
wherein the error estimator is configured to estimate a classification error between the classification label predicted by the learning model and a ground-truth label included in a labeled image.

19. The computer-implemented method of claim 12, wherein the image analysis task is an object detection task, the learning model is configured to predict a bounding box surrounding an object and a classification label of the object,
wherein the error estimator is configured to estimate a localization error between the predicted bounding box and a ground-truth bounding box included in a labeled image, or a classification error between the classification label predicted by the learning model and a ground-truth label included in the labeled image.

20. A non-transitory computer-readable medium having a computer program stored thereon, wherein the computer program, when executed by at least one processor, performs a method for analyzing medical images using a learning model, the method comprising:
receiving a medical image acquired by an image acquisition device; and
applying the learning model to perform an image analysis task on the medical image,
wherein the learning model is trained jointly with an error estimator using training images comprising a first set of labeled images and a second set of unlabeled images, wherein the error estimator is configured to estimate an error of the learning model associated with performing the image analysis task,
